(19) United States
(12) Patent Application Publication
(10) Pub. No.: US 2026/0102579 A1
(43) Pub. Date: Apr. 16, 2026

(54) APPARATUSES AND METHODS TO PRODUCE HEATED AIR

(71) Applicant: Respiratory Health Technologies, Inc., Indianapolis, IN (US)

(72) Inventors: Reuben Quincey Zielinski, Fishers, IN (US); Robert Anderson Till, JR., Avon, IN (US); Jackson Alexander Townsend, Indianapolis, IN (US); Matthew Raymond Zielinski, Cleveland Heights, OH (US)

(73) Assignee: Respiratory Health Technologies, Inc., Indianapolis, IN (US)

(21) Appl. No.: 19/360,700

(22) Filed: Oct. 16, 2025

Related U.S. Application Data
(60) Provisional application No. 63/707,856, filed on Oct. 16, 2024.

(51) Int. Cl. A61M 16/10; A61L 2/00; A61L 2/06; A61L 2/26; F24H 3/04

(52) U.S. Cl. CPC A61M 16/1075; A61L 2/04; A61L 2/06; A61L 2/26; A61L 2103/05; A61M 16/1005; A61M 16/106; F24H 3/0405; A61M 2205/362; F24H 2250/02

(57) ABSTRACT

A heated air generator includes: a fan configured to generate a flow of gas; a housing coupled to the fan and including an intake port positioned to allow the flow of gas to enter the housing and an exhaust port positioned to allow the flow of gas to exit the housing; and a heat generator disposed within the housing and including a plurality of heating arrays that are stacked and positioned such that the flow of gas impinges on the heating arrays between the intake port and the exhaust port before exiting the housing.

### CROSS REFERENCE TO RELATED APPLICATIONS

This is a non-provisional application based on U.S. Provisional Patent Application Ser. No. 63/707,856 entitled “APPARATUSES AND METHODS TO PRODUCE HEATED AIR,” filed on Oct. 16, 2024, which is incorporated herein by reference.

### BACKGROUND OF THE INVENTION

### 1. Field of the Invention

This invention relates generally to apparatuses and methods to produce heated air for the reduction of virus plaque forming units which are normally associated with the common cold (Rhinovirus), Severe Acute Respiratory Syndrome (SARS), and Coronavirus Disease 2019 (COVID-19).

### 2. Description of the Related Art

According to the Centers for Disease Control and Prevention (CDC), many viruses can cause produce standard common cold symptoms such as rhinitis and congestion, but rhinoviruses are the most common and hence, are most often associated with the common cold. These rhinoviruses are spread through respiratory droplets and close personal contact. Accordingly, it has been generally understood because of the prevalence of these rhinoviruses, there is no cure for the common cold and society has primarily focused on prevention methods for common cold reduction. These prevention methods include washing hands thoroughly with soap and water for as long as 20 seconds, disinfecting surfaces, avoiding people with colds, and avoiding touching eyes, nose, and mouth with unwashed hands. Because the virus incubates within the superficial epithelium of the mucosal surfaces of the oropharynx and nasopharynx it is easily transmitted.

By current estimates, in the course of a year, people in the U.S. suffer over 1 billion colds. Most colds occur during the fall and winter months. Beginning in late August or early September, the rate of colds increases slowly for a few weeks and remains high until March or April, when it begins to decline. The seasonal variation may relate to the opening of schools and to cold weather, which prompts people to spend more time indoors and increases the chances that viruses will spread between personal contacts. During these cold seasons, according to some estimates, over $40 billion is spent in the U.S. on cold remedies.

Children usually have about 6 to 10 colds per year. One important reason why colds are so common in children is because they are often in close contact with each other in daycare centers and schools. In families with children in school, the number of colds per child can be as high as 12 per year. Adults average about 2 to 4 colds per year, although the range varies widely. Women, especially those aged 20 to 30 years, have more colds than men, possibly because of their closer contact with children. On average, people older than age 60 have fewer than one cold per year.

Seasonal changes in relative humidity also may affect the prevalence of colds. The most common cold-causing viruses survive better when humidity is low and during the colder months of the year. Cold weather also may make the inside lining of your nose drier and more vulnerable to viral infection. Sneezing, scratchy throat, runny nose are the first signs of a cold, and probably the most common illness known. Although the common cold is usually mild, with symptoms lasting 1 to 2 weeks, it is a leading cause of doctor visits and missed days from school and work. According to the CDC, 22 million school days are lost annually in the United States because of the common cold.

It is common knowledge that many people, during warmer, more humid months, begin to feel the symptoms of the common cold as they enter and exit air-conditioned premises and offices. Many times, people complain they get scratchy and sore throats just from spending time in their air-conditioned cars. Sleeping under a ceiling fan or next to an open window with the night air dropping often leads to common cold systems.

Human rhinovirus is the most common cause of the common cold. It replicates in the superficial mucosa of the nose and throat. The inflammatory response to the virus causes the symptoms, and the virus replicates optimally at 33 degrees Celsius. The replication of the virus is impeded by increasing temperatures. The decrease in viral replication appears to be directly correlated to an increase in temperature.

The human rhinovirus was discovered to be the causative agent of the respiratory illness moving through the Baltimore Hospitals and John Hopkin's Hospital in the 1950's. Additional studies isolated the human rhinovirus from monkeys and horses in 1956 and concluded it produced an upper airway infection leading to malaise, coryza (inflammation of the nose), and a mild sore throat. Rhinoviruses are the most commonly recovered agents from people with mild upper respiratory illnesses. They are usually isolated from nasopharyngeal secretions but may also be found in throat and oral secretion. Replication is limited to the surface epithelium of the nasal mucosa.

It is currently believed that the two most common symptoms, rhinorrhea and nasal obstruction, are a result of the increased neutrophilic inflammatory response to the virus. There is an increased permeability in the mucous membranes of the nasopharynx in response to the virus. This leads to an increase in the production and excretion of mucus. Many believe that the cough that often develops later in the illness results from the excess drainage of mucus into the posterior pharynx.

There are some lower respiratory symptoms seen in asthma patients but the mechanism behind the symptoms is still debated. Additionally, scientists still debate whether the virus can infect cells of the lower respiratory track. In the 1930's, Tyrell and Rodgers discovered that the human rhinovirus optimally grows at 33 degrees Celsius, or 91.4 degrees Fahrenheit.

There is a need, therefore, for a new technique and associated apparatus which produces heated air efficiently and quickly to reduce the growth of viruses and addresses one or more of the shortcomings of the treatments for the common cold, SARS, COVID-19, and any other viruses that can be attenuated with higher temperatures according to the prior art.

### SUMMARY OF THE INVENTION

At least one of the embodiments described herein includes an apparatus and method having features that provide better, more consistent heated air production for reducing viruses as well as a simple way to measure the treatment time.

A first embodiment is a heated air generator having at least a first flexible circuit heater board, an integrated controller within the flexible circuit heater board, and a fan actuated air pressure producer. The heater board includes a plurality of surface mount resistors configured in multiple annulus patterns which are folded to fashion a heated stack.

A second embodiment of a heated air generator that includes a plastic injection molded center mount used for wrapping a flexible circuit around, and center mount covers for both proximal and distal ends of said center mount and flexible circuit. The center mount provides air pathways which route air in and around the plurality of surface amount resistors mounted in an annulus, with the proximal and distal covers providing further vectoring of air upon intake and exhaust.

In some embodiments provided according to the present invention, a heated air generator includes: a fan configured to generate a flow of gas; a housing coupled to the fan and including an intake port positioned to allow the flow of gas to enter the housing and an exhaust port positioned to allow the flow of gas to exit the housing; and a heat generator disposed within the housing. The heat generator includes heating arrays that are stacked and positioned such that the flow of gas impinges on the heating arrays between the intake port and the exhaust port before exiting the housing.

### BRIEF DESCRIPTION OF THE DRAWINGS

Some of the figures shown herein may include dimensions or may have been created from scaled drawings. However, such dimensions, or the relative scaling within a figure, are by way of example only, and not to be construed as limiting the scope of this invention. In the drawings:

FIG. 1 illustrates a perspective view of a heated air generator assembly;

FIG. 2 illustrates a top view of flexible printed circuit assembly used to generate heat in the heated air generator;

FIG. 3 illustrates a perspective view of a heated air generator subassembly of FIG. 1 with flexible circuit and central air vectoring mount;

FIG. 4 illustrates an exploded perspective view of the heated air generator subassembly of FIG. 3 with exhaust vector air cover;

FIG. 5 illustrates a perspective view of the heated air generator subassembly of FIG. 4 with flexible circuit folded and exhaust vector air cover attached;

FIG. 6 illustrates a perspective view of the heated air generator subassembly of FIG. 5 with flexible circuit folded and intake vector air cover attached;

FIG. 7 illustrates a cutaway perspective view of the heated air generator assembly of FIG. 1 with air flow paths as a result of air vectoring features;

FIG. 8 illustrates a perspective view of the central air vectoring mount of FIG. 3;

FIG. 9 illustrates an outside surface perspective view of the exhaust vector air cover of FIG. 4; and

FIG. 10 illustrates an inside surface perspective view of the exhaust vector air cover of FIG. 4.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

For the purposes of promoting an understanding of the principles of the invention, reference is made to selected embodiments illustrated in the drawings and specific language will be used to describe the same. It will nevertheless be understood that no limitation of the scope of the invention is thereby intended; any alterations and further modifications of the described or illustrated embodiments, and any further applications of the principles of the invention as illustrated herein are contemplated as would normally occur to one skilled in the art to which the invention relates. At least one embodiment of the invention is shown in greater detail, although it will be apparent to those skilled in the relevant art that some features or some combinations of features may not be shown for the sake of clarity.

Any reference to “invention” within this document is a reference to an embodiment of a family of inventions, with no single embodiment including features that are necessarily included in all embodiments, unless otherwise stated. Furthermore, although there may be references to “advantages” provided by some embodiments of the present invention, other embodiments may not include those same advantages or may include different advantages. Any advantages described herein are not to be construed as limiting to any of the claims.

Specific quantities (spatial dimensions, temperatures, pressures, times, force, resistance, current, voltage, concentrations, wavelengths, frequencies, heat transfer coefficients, dimensionless parameters, etc.) may be used explicitly or implicitly herein, such specific quantities are presented as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated.

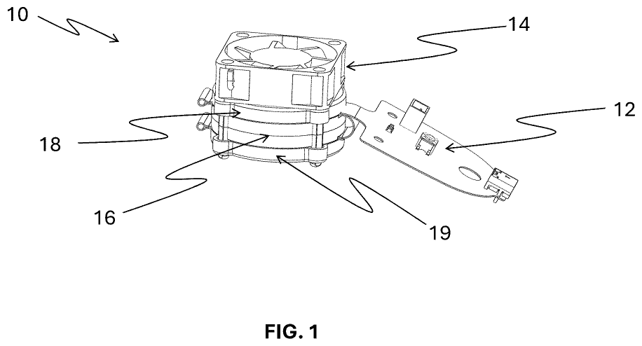

Referring now to the drawings, FIG. 1 shows a perspective side view of a first exemplary embodiment of a heated air generator 10 comprising an integrated control and heater flex circuit 12, pressure generating fan 14, central air vectoring mount 16 (which may be simply called a “central mount”), intake air cover 18 and exhaust air cover 19. The heated air generator 10, which may also be referred to as a heating arrangement, may be sized and shaped to be incorporated in and/or coupled to a respiratory therapy device, such as a breathing mask, in order to provide heated gas to a treatment subject and heat the upper respiratory tract of the subject. In some embodiments, the heated air generator 10 is disposed inside a breathing mask, which acts as a full or partial housing, so gas brought into the breathing mask through one or more intakes gets heated by the heated air generator 10 before being inhaled by a treatment subject that is wearing the mask. The one or more intakes of the breathing mask may include, for example, a filter that is configured to reduce the number of particles that the treatment subject will inhale while wearing the breathing mask. It should be appreciated that, in some embodiments, the heated gas is air, such as ambient air, but it should also be appreciated that the breathing mask may be coupled to a gas tank containing a gas mixture including a higher percentage of oxygen (and/or other gas(es)) than ambient air. The heated air generator 10 can thus be incorporated in a breathing mask to provide stand-alone heated gas respiratory therapy or used in combination with other respiratory therapy techniques such as increased oxygen therapy.

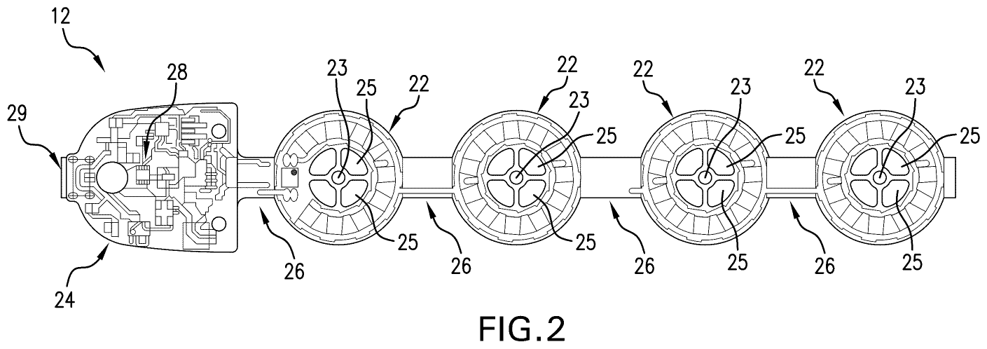

As best shown in FIG. 2, integrated control and heater flex circuit 12 includes control circuitry 24, surface mount resistor arrays 22, which may also be referred to as “heating arrays,” are soldered to a substrate of the integrated control and heater flex circuit 12, bendable connecting pathways 26, all fabricated from flexible copper circuit path plating that is 0.5 ounce/ft2-1.5 ounce/ft2 and allows ample heat conduction and current to power surface mount resistors of the arrays 22. The circuit 12 may also be referred to simply as a “heat generator” herein. The substrate of the integrated control and heater flex circuit 12 is thin enough to be flexible so the circuit 12 can flex in the illustrated manner without disrupting the conductive pathways. In some embodiments, integrated control and heater flex circuit 12 is a two-layer circuit with copper pours between the circuit traces. Copper pours act as thermal heat sinks thus allowing the entire surface to heat uniformly. Surface mount resistor arrays 22 are attached in the form of an annulus pattern which allows air to be impinged on each individual surface mount resistor of the arrays 22. The resistor arrays 22 each surround a center hole 23 formed in the substrate and at least one air flow opening 25 formed in the substrate that is disposed between the arrays 22 and the respective center hole 23 to allow air flow therethrough. The number and configuration of the resistors of the resistor arrays 22 may be chosen so the resistor arrays 22 heat gas, such as air, flowing over the resistor arrays 22 to a temperature of between 85° F.-200° F. before the air exits the generator 10. While the heating arrays 22 are described and illustrated as being arrays of resistors that are configured to generate heat from electrical current, it should be appreciated that the heating arrays can take other forms, such as chemical heating arrays that utilize chemical reactions to form heat. In some embodiments, the control circuitry 24 may be configured to sense the temperature of gas exiting the heated air generator 10 (and/or the breathing mask) and adjust electrical power delivery to the resistor arrays 22 accordingly so the gas exiting the heated air generator 10 stays within a defined temperature range, which may be between 85° F.-200° F., such as between 95° F.-110° F.

Control circuitry 24 of integrated control and heater flex circuit 12 has a controller in the form of a standard USB-C port controller 28 which allows voltage selection of 20V from standard USB-C power blocks via USB-C receptable 29. It should be appreciated that while the control circuitry 24 is illustrated and described as having a standard USB-C port controller 28 and a standard USB-C receptacle, other types of electrical power port controllers and receptacles can be incorporated in the control circuitry 24 and do not necessarily need to meet any particular electronic power standards. USB-C port controller 28 is configured for 20V at 2-3 amperes. Although USB-C controller 28 is configured at 20V 2-3 amperes, in other embodiments USB-C port controller could be configured at 5V, 9V, 12V, or 15V. In some embodiments, surface mount resistor arrays 22 produce 25W-45W for rapid, adequate heating of air. In some embodiments, USB-C port controller 28 is configured for 20V supply to maintain a lower current draw for 25-40W heating.

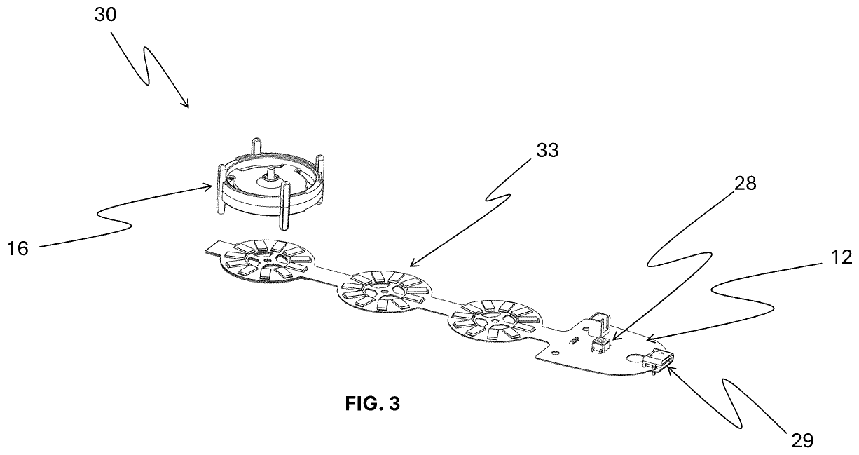

Referring now to FIG. 3, center mount flex sub-assembly 30 is depicted with integrated control and heater flex circuit 12 of FIG. 2 and center mount 16 prior to integrated control and heater flex circuit 12 of FIG. 2 being folded onto center mount 16. In other words, FIG. 3 illustrates the control and heater flex circuit 12 in an unfolded orientation. In the unfolded orientation, some or all of the mount resistor arrays 22 may generally be in-line with one another and be situated in the same plane, assuming that the thicknesses of the arrays 22 are the same. If the thicknesses of the arrays 22 differ, the bottoms of the substrate on which the arrays 22 are disposed may be situated in the same plane in the unfolded orientation. It should be appreciated that two arrays 22 may be disposed on opposite sides of the substrate in the unfolded orientation.

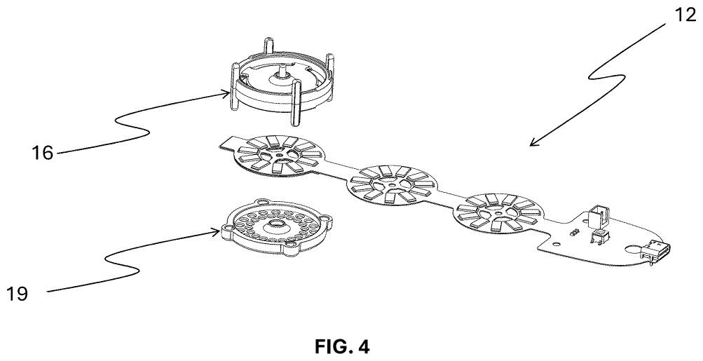

As best shown in FIG. 4, integrated control and heater flex circuit 12 of FIG. 2 is depicted with central air vectoring mount 16 of FIG. 1 and exhaust cover 19 of FIG. 1 aligned for mounting and folding of integrated control and heater flex circuit 12 of FIG. 2. The control and heater flex circuit 12 is depicted in the unfolded orientation in FIG. 4 as well.

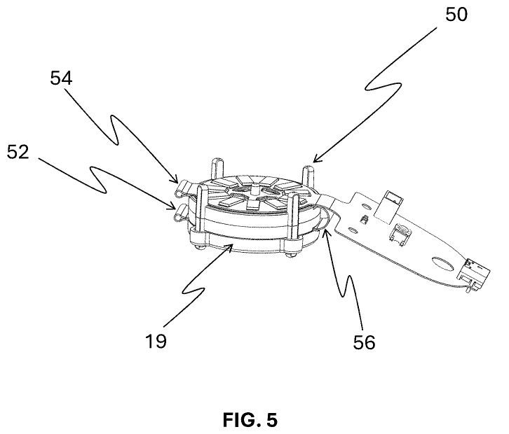

Referring now to FIG. 5, center mount flex subassembly 30 of FIG. 3 is depicted with exhaust cover 19 of FIG. 1 assembled using alignment pins 50. The control and heater flex circuit 12 has been folded to a folded orientation so the resistor arrays 22 are no longer situated in the same plane but rather are stacked relative to one another. As used herein, the arrays 22 are “stacked” in the sense that the arrays 22 are disposed vertically one on top of another, as illustrated. In some embodiments, each of the arrays 22 defines a respective plane that, when stacked, are parallel to the plane(s) of the other arrays 22 so the planes of all of the arrays 22 are parallel. Flex circuit bendable and connecting pathways 26 of FIG. 2 are shown folded, creating folds 52, 54, and 56.

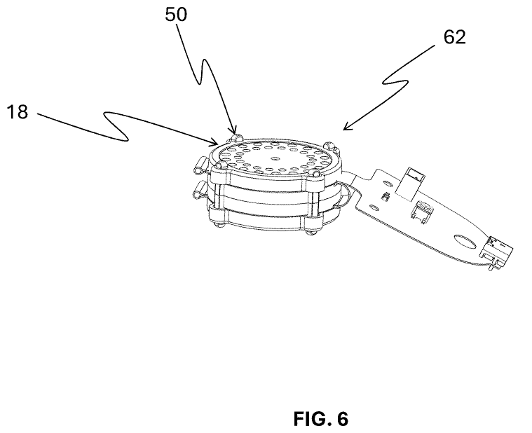

As best shown in FIG. 6, intake cover 18 of FIG. 1 is aligned and press-fit to the central mount 16 using alignment pins 50 of FIG. 5. Press-fitting, i.e., coupling, the intake cover 18 to the central mount 16 on a first side and the exhaust cover 19 to the central mount 16 on a second side opposite the first side creates a housing to enclose the heat generator 12 including the resistor arrays 22 so they are not exposed, reducing the risk of a user touching any of the resistors and burning themselves and/or damaging the resistors.

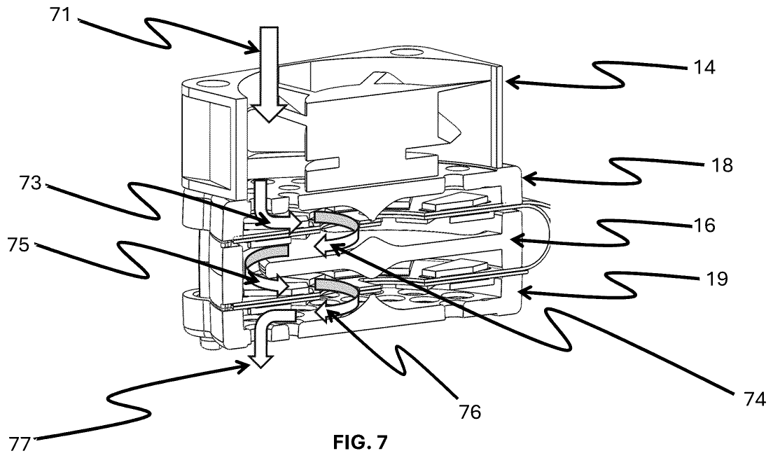

In some embodiments, airflow paths of FIG. 7 can be achieved using ports of central air vectoring mount 16, intake cover 18, and exhaust cover 19 all of FIG. 1. Intake air 71 is drawn in through pressure generating fan 14 of FIG. 1 and is forced through ports of intake cover 18 so the air, shown as airpath 73, impinges on at least one of the surface mount resistor arrays 22 disposed in a first compartment formed between the intake cover 18 and the central mount 16. Air enters through openings in central air vectoring mount 16 of FIG. 1 producing airpath 74. Airpath 74 travels over second surface mount resistor arrays 22 of FIG. 2 producing airpath 75. Airpath 75 travels into a second compartment formed between the central mount 16 and the exhaust cover 19 in which at least one of the surface mount resistor arrays 22 is disposed producing airpath 76, which in turn travels over fourth surface mount resistor arrays 22 of FIG. 2 producing airpath 77 which exits exhaust cover 19. In this respect, the intake air 71 impinges on at least a portion of each of the stacked multiple resistor arrays 22 as it travels through the assembled housing from the intake cover 18 and out the exhaust cover 19. The intake air 71 thus travels over at least a portion of each of the stacked resistor arrays 22 disposed in the housing for heating, which provides a compact assembly for heating the air before it exits through the exhaust cover 19 to, for example, a breathing mask for inhalation by a user.

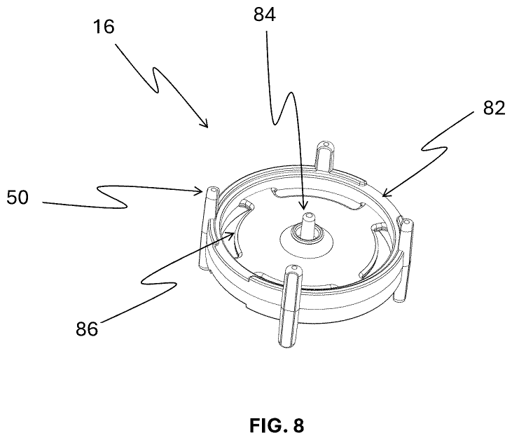

Referring now to FIG. 8, the central air vectoring mount 16 is shown with flex circuit alignment channel 82, center alignment pin 84, at least one vector air flow channel shown in the form of multiple vector air flow channels 86, and alignment pins 50. Center alignment pin 84 is pushed through respective center holes 23 that are each surrounded by surface mount resistor arrays 22 of FIG. 2, with bendable connecting pathways 26 being placed into flex circuit alignment channel 82. Central air vectoring mount 16 is designed to hold, align, and channel air over integrated control and heater flex circuit 12 of FIG. 2. The vector air flow channel(s) 86 is formed in the central mount 16 to direct the flow of gas therethrough. In this respect, the size and shape of the vector air flow channels 86 can be adjusted as desired to change the flow characteristics of air within the housing past the central air vectoring mount 16. As illustrated, the channels 86 extend circumferentially between an outer ring to which the alignment pins 50 are connected and an inner ring that supports the resistor array 22. It should be appreciated that the channels 86 can take other shapes and sizes, e.g., as openings formed in the inner ring.

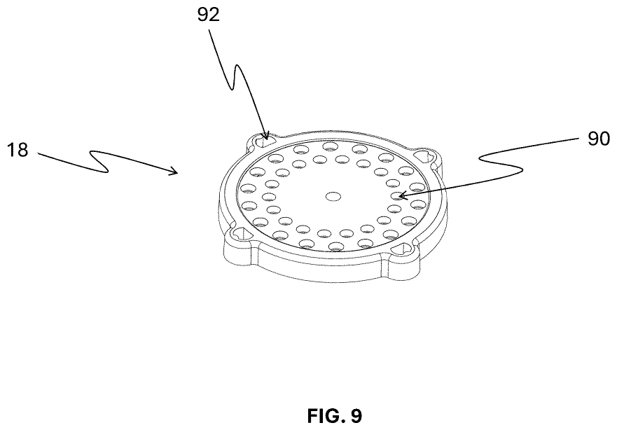

As best shown in FIG. 9, exhaust cover 19 is depicted from the outside surface with alignment pin receptacle 92 and exhaust air discharge ports 90. In some embodiments, exhaust air discharge ports 90 are similar in size and are arranged to fit over surface mount resistor array 22 of FIG. 2. In some embodiments, exhaust air discharge ports 90 are arranged circumferentially and crowded to the edge of exhaust cover 19 to enable the air to flow over surface mount resistors of FIG. 2, thus picking up convective heat and discharging this heated air through exhaust discharge ports 90.

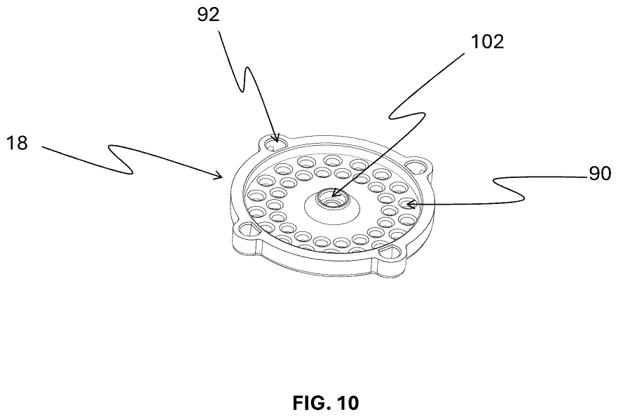

Now referring to FIG. 10, exhaust cover 19 is depicted from the inside surface with center axis alignment receptacle 102 together with exhaust air discharge ports 90 and alignment pin receptacle 92 of FIG. 9. Exhaust cover 19 fits over central air vectoring mount 16 with center alignment pin 84 of FIG. 8 fitting into center axis alignment receptacle 102. Intake air cover 18 of FIG. 1 is symmetrical to exhaust cover 19 with air flowing in the opposite direction as depicted in FIG. 7.

In some embodiments, central air vectoring mount 16 of FIG. 1 provides an assembly platform utilizing integrated control and heater flex circuit 12 of FIG. 2. Central air vectoring mount 16 of FIG. 8 is symmetrical and has center alignment pin 84 on each side. Surface mount resistor arrays 22 of FIG. 2 each have a respective center hole 23 which fits over center alignment pin 84 of FIG. 8. Utilizing the two innermost surface mount resistor arrays 22 of FIG. 2, the integrated control and heater flex circuit 12 is wrapped over each side of central air vectoring mount 16 and placed on center alignment pin 84. The outermost surface mount resistor arrays 22 are now folded back over the innermost surface mount resistor arrays 22 and pushed onto center alignment pin 84 on each side of central air vectoring mount 16. Flex circuit bendable and connecting pathways 26 of FIG. 2 are now folded into flex circuit alignment channel 82 of FIG. 8. Exhaust cover 19 and intake cover 18 of FIG. 1 are now placed over surface mount resistor arrays 22 on each side of central air vectoring mount 16 and pressed together so alignment pins 50 are disposed in respectively aligned alignment pin receptacles 92. This stacks and holds the resistor arrays 22 together, providing a compact heating arrangement.

In some embodiments, the control circuitry 24 is configured to independently control the amount of power that is delivered to and utilized by each of the resistor arrays 22. In some embodiments, the control circuitry 24 is configured to interface with external control circuitry, such as control circuitry of a breathing mask apparatus, to control the amount of power that is delivered to and utilized by each of the resistor arrays 22 in tandem with the external control circuitry. In this respect, the heated air generator 10 can be a “plug and play” device that is easily integrated into a variety of devices for generating heated air. It should thus be appreciated that the heated air generator provided according to the present invention can be used to deliver heated air in a variety of devices and not just breathing masks.

The present invention also provides a method of generating heated air using the heated air generator 10. The method includes forcing gas, such as air, through the intake cover 18 into the housing using the fan 14 and heating the gas in the housing using the heating arrays 22. The heated gas then exits the housing via the ports 90 of the exhaust cover 19 and, in some embodiments, the method further includes delivering the heated gas to a patient via, for example, a breathing mask or other device.

From the foregoing, it should be appreciated that the heated air generator 10 provided according to the present invention provides a flexible and compact assembly for generating heated air at controlled temperatures. The temperature of the heated air that is produced can be adjusted in a variety of ways, including but not limited to adjusting the rotation speed of the fan 14 to adjust the amount of intake air 71 that enters the generator 10 and/or by adjusting the temperature of one or more of the resistor arrays 22 that contact the intake air 71 before it exits through the exhaust cover 19. The assembly is compact due to stacking the resistor arrays 22 and it should be appreciated that the number of resistor arrays 22 that are stacked can be adjusted, as desired, to adjust the amount of thermal energy that is delivered to the intake air 71, e.g., more resistor arrays 22 can be incorporated to produce hotter air and fewer can be incorporated if less hot air is desired. Thus, the heated air generator 10 provided according to the present invention reliably provides heated air of various temperatures in a compact arrangement that can be incorporated into a variety of devices.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A heated air generator, comprising:
a fan configured to generate a flow of gas;
a housing coupled to the fan and comprising an intake port positioned to allow the flow of gas to enter the housing and an exhaust port positioned to allow the flow of gas to exit the housing; and
a heat generator disposed within the housing and comprising a plurality of heating arrays that are stacked and positioned such that the flow of gas impinges on the heating arrays between the intake port and the exhaust port before exiting the housing.

2. The heated air generator of claim 1, wherein at least one of the heating arrays comprises a resistor array configured to generate heat from electrical current.

3. The heated air generator of claim 1, wherein the housing comprises an intake cover, a central mount coupled to the intake cover on a first side, and an exhaust cover coupled to the central mount on a second side opposite the first side.

4. The heated air generator of claim 3, wherein a first compartment is formed between the intake cover and the central mount and a second compartment is formed between the central mount and the exhaust cover, at least one of the heating arrays being disposed in the first compartment and at least one of the other heating arrays being disposed in the second compartment.

5. The heated air generator of claim 4, wherein the central mount has at least one vector air flow channel formed therein to direct the flow of gas therethrough.

6. The heated air generator of claim 5, wherein the at least one vector air flow channel extends circumferentially.

7. The heated air generator of claim 5, wherein the heating arrays are formed as part of a circuit comprising a substrate on which all of the heating arrays are disposed and bendable conductive pathways connected to the heating arrays.

8. The heated air generator of claim 7, wherein the circuit is bent such that the heating arrays are stacked within the housing.

9. The heated air generator of claim 8, wherein the heating arrays are stacked such that the flow of gas impinges on at least a portion of each of the heating arrays disposed within the housing as the flow of gas travels from the intake port out the exhaust port.

10. The heated air generator of claim 8, wherein the central mount comprises a center alignment pin and each of the heating arrays is disposed on the substrate to surround a respective center hole formed in the substrate that is placed on the center alignment pin to bend the circuit and stack the heating arrays.

11. The heated air generator of claim 10, wherein each of the heating arrays comprises a plurality of resistors arranged on the substrate in an annulus pattern surrounding the respective center hole.

12. The heated air generator of claim 11, wherein the substrate has at least one air flow opening formed therein between each of the heating arrays and the respective center hole.

13. The heated air generator of claim 3, wherein the intake cover and the exhaust cover each comprise a plurality of ports formed therein to allow the flow of gas therethrough.

14. The heated air generator of claim 13, wherein the intake cover is symmetrical to the exhaust cover.

15. The heated air generator of claim 14, wherein the intake cover and the exhaust cover each comprise a plurality of alignment pin receptacles and the central mount comprises a plurality of alignment pins that are each disposed in respectively aligned alignment pin receptacles.

16. The heated air generator of claim 1, wherein the heat generator comprises a substrate on which the heating arrays are disposed, the heat generator having an unfolded orientation where the heating arrays are all situated in a same plane and a folded orientation where the heating arrays are all stacked relative to one another.

17. The heated air generator of claim 1, further comprising control circuitry that is electrically coupled to the heat generator and configured to control voltage delivery to the heat generator.

18. The heated air generator of claim 17, wherein the control circuitry comprises a universal serial bus type C (USB-C) port controller.
